(19) United States
(12) Patent Application Publication
(10) Pub. No.: US 2025/0325877 A1
(43) Pub. Date: Oct. 23, 2025

(54) Method to Identify Right and Left Impact during Running using a Single Sacrum-Mounted IMU

(71) Applicant: University of Oregon, Eugene, OR (US)

(72) Inventors: Michael Hahn, Eugene, OR (US); Aida Chebbi, Eugene, OR (US); Rachel Robinson, Eugene, OR (US); Seth Donahue, Lexington, KY (US)

(21) Appl. No.: 19/182,423

(22) Filed: Apr. 17, 2025

Related U.S. Application Data
(60) Provisional application No. 63/635,969, filed on Apr. 18, 2024.

(51) Int. Cl. A63B 24/00; G01C 21/16

(52) U.S. Cl. CPC A63B 24/0062; G01C 21/16; A63B 2024/0071; A63B 2220/22; A63B 2220/40; A63B 2220/803; A63B 2220/836

(57) ABSTRACT

A method for biomechanical motion tracking records accelerometer and rate gyroscope data from a single inertial measurement unit mounted on the sacrum of a runner. Recorded resultant linear acceleration measurements and recorded angular velocity measurements about the anterior-posterior axis are processed to identify right foot impact events and left foot impact events. Impact events are identified using crackle, the third derivative of the resultant linear acceleration measurements. Left and right impact events are distinguished using the angular velocity measurements.

### CROSS-REFERENCE TO RELATED APPLICATIONS

This application claims priority from U.S. Provisional Patent Application 63/635,969 filed Apr. 18, 2024, which is incorporated herein by reference.

### STATEMENT OF FEDERALLY SPONSORED RESEARCH

None.

### FIELD OF THE INVENTION

The present invention relates generally to systems and methods for determination of biomechanical motion. More specifically, it relates to techniques for gait analysis and accurately identifying foot impact events during running.

### BACKGROUND OF THE INVENTION

Gait analysis, a comprehensive examination of human locomotion, is foundational in various fields such as sports science, rehabilitation, and clinical diagnostics. When analyzing running performance and injury prevention, identifying gait events such as initial contact (IC) and final contact, commonly called toe off (TO), for each foot is crucial. Traditional methods often involve complex setups with multiple sensors, cameras, force plates, and instrumented treadmills, which can influence natural movement patterns.

The advent of wearable sensors, particularly inertial measurement units (IMUs), has revolutionized gait analysis by offering an affordable and practical alternative to traditional three-dimensional motion capture systems (3D MOCAP). These wearable sensors can continuously record data during prolonged runs in natural environment. When placed on the foot dorsum, IMU data such as linear acceleration can be used to calculate stride and step length, cadence stance time and we can estimate ground reaction force. When placed on the waistband, close to the sacrum, IMU data such as angular velocity can be used to calculate pelvic tilt, pelvic rotation, running symmetry. Having multiple sensors on various parts of the body, however, is complex and inconvenient.

Attempts have been made to use a single sensor located at the sacrum for gait analysis. These attempts, however, are limited to controlled level ground (LG) track and treadmill conditions, and are not sufficiently accurate in a variety of unpredictable running conditions. Disruptions can include strains or sprains from sudden movements or overuse, and quick lateral movements to dodge obstacles. These incidents can alter the runner's pace and stride, adding complexity to the cyclic nature of running. These unpredictable and uncontrolled conditions of data collection pose significant challenges for accurately analyzing the data to correctly and reliably identify and distinguish right and left foot strikes.

### SUMMARY OF THE INVENTION

The techniques disclosed herein are able to accurately identify and distinguish between the right and left foot impact during running not only on level ground, but also on incline, and decline across various speeds. In contrast to the majority of commercial products on the market that require at least two IMUs, this technique is able to accomplish accurate left-right impact identification using a single Inertial Measurement Unit (IMU) placed on the sacrum. This improved accuracy and reliability of identifying gait events is crucial in many applications such as clinical gait analysis, sports performance assessment, and the development of assistive devices.

In one aspect, the present invention provides a method for detecting right and left foot strike events during running. A signal processing approach uses high order derivatives of signals from inertial measurement units. Specifically, the method uses the ‘crackle’ function, the third derivative of acceleration, which offers more precise and accurate identification of impact events compared to previous methods. This approach is particularly effective in identifying the initial peak in acceleration data during foot impact with the ground. The method involves detecting the critical points where the crackle function is zero, indicating potential peaks or troughs. These points correspond to maximum changes in acceleration, which are indicative of important events like foot strikes or toe-offs. The algorithm then locates the closest peak in the linear acceleration data, which corresponds to the beginning of impact. To determine whether the impact is from the right or left foot, the algorithm searches for the corresponding peak or trough in the frontal plane angular velocity within a window of 5 ms around the linear acceleration peak. If the angular velocity peak is positive, it indicates a right foot impact, and vice versa for a left foot impact.

The method uses crackle in combination with the angular velocity about the anterior-posterior axis to accurately detect left and right foot impact from a single IMU placed on the sacrum during graded running. The method assesses both the resultant acceleration and the angular velocity of a sacral-mounted sensor, providing a more comprehensive analysis of foot impacts with a single IMU. Unlike previous methods that were conducted in laboratory settings, this new method can be applied in real-time during actual running sessions.

This method is valid not only in controlled level ground running but also on incline and decline conditions, and various other unpredictable and uncontrolled running conditions. The method accurately identifies right and left side impacts during level ground, incline and decline runs across a range of speeds. The method is experimentally compared with other existing methods to demonstrate its superior effectiveness. The ability to attain such accuracy with a single sensor is particularly beneficial in competitive events such as marathons where the minimalistic setup can prevent any restraint to the runner's performance. In contrast with existing techniques, this method is adaptable to changes in running patterns, making it suitable for real-world outdoor running scenarios.

Another advantage of this technique is that its analysis is localized in time and does not depend on an analysis of lengthy waveforms. Each impact event is analyzed independently of the entire waveform recorded during the trial. If any mistakes are made with the detection of previous peaks, that mistake does not affect the current instance. This overcomes a common issue in previous approaches which rely on the position of previously detected events to correctly analyze a current event.

The IMU may be integrated with the processor, e.g., a smartphone with integrated IMU can perform measurements and processing. The smartphone could be attached to the sacrum using a belt or other mechanism. The IMU could also be a separate device attached to the sacrum that communicates the IMU data in real time to a separate processing device via a wireless link (e.g., Bluetooth). Alternatively, the IMU device could record the data during running and transfer the recorded data later to a separate processing device.

In one aspect, the invention provides a method for biomechanical motion tracking, the method comprising: a) recording accelerometer and rate gyroscope data from an inertial measurement unit mounted on the sacrum of a runner, wherein the accelerometer data comprises resultant linear acceleration measurements and the rate gyroscope data comprises angular velocity measurements about the anterior-posterior axis; b) processing the recorded accelerometer data to identify right foot impact events and left foot impact events, wherein the processing comprises identifying impact events using the third derivative of the resultant linear acceleration measurements, and distinguishing between left and right impact events using the angular velocity measurements about the anterior-posterior axis, wherein the processing uses acceleration and angular velocity data from only the inertial measurement unit mounted on the sacrum of the runner. Preferably, identifying impact events using the third derivative of the resultant linear acceleration measurements comprises using maxima of the third derivative of the resultant linear acceleration measurements to determine locations of impact peaks in the resultant linear acceleration that are closest temporally to the locations of the crackle maxima. Preferably, distinguishing between left and right impact events comprises searching for extrema in the frontal plane angular velocity within a window of 5 ms around an identified impact event, and identifying the impact event as a right foot impact or left foot impact depending on whether the angular velocity peak is positive or negative. The recording and processing steps may be performed by a single portable electronic device or may be performed by two distinct portable electronic devices in which case the recorded accelerometer and rate gyroscope data are communicated between the two distinct portable electronic devices. The method may further include processing the recorded accelerometer and rate gyroscope data, right foot impact events, and left foot impact events to provide analysis of gait patterns. The method may further include processing the recorded accelerometer and rate gyroscope data, right foot impact events, and left foot impact events to provide analysis of athletic technique.

### BRIEF DESCRIPTION OF THE DRAWINGS

FIG. 1A is a schematic diagram showing a single inertial measurement unit mounted near the sacrum of a runner, according to an embodiment of the invention.

FIG. 1B is a schematic diagram illustrating components of an inertial measurement unit used to perform the method according to an embodiment of the invention.

FIG. 2 is a flowchart outlining the main steps of a method for identifying right and left impact events using an inertial measurement unit according to an embodiment of the invention.

FIG. 3 is a graph of a resultant acceleration function and corresponding crackle function illustrating the use of extrema in the crackle function to locate peaks in the resultant acceleration function that represent impact events, according to an embodiment of the invention.

FIG. 4 is a graph of a resultant acceleration function and corresponding angular velocity function illustrating the use of extrema in the angular velocity function to distinguish whether peaks in the resultant acceleration function represent right or left foot impact events, according to an embodiment of the invention.

### DETAILED DESCRIPTION OF THE INVENTION

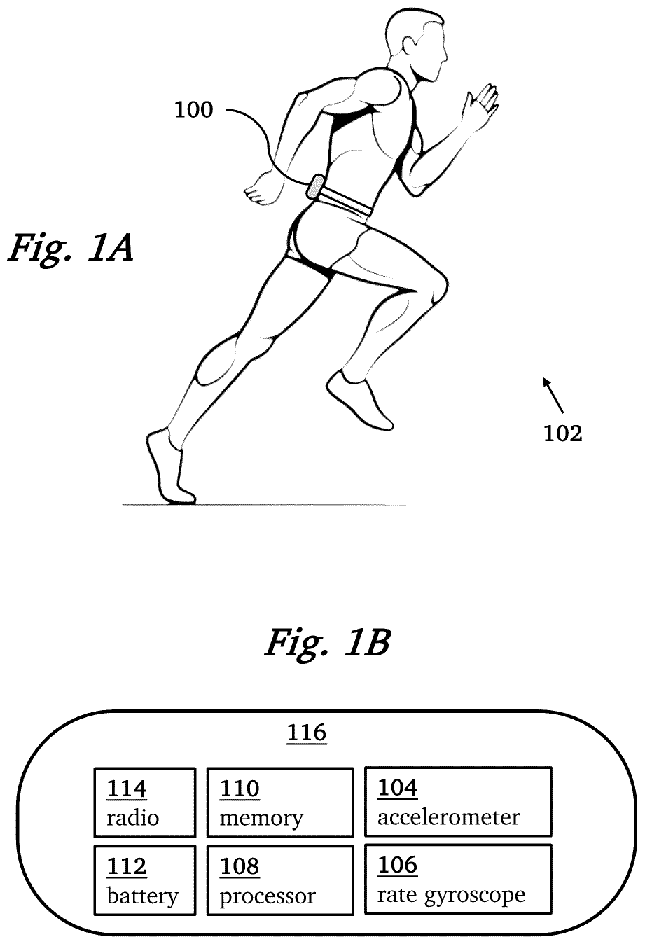

According to one embodiment of the invention, as illustrated in FIG. 1A, a single Inertial Measurement Unit (IMU) 100 placed on the sacrum of a person running 102 is used to perform gait analysis. This method is valid not only on level ground but also in various unpredictable and changing conditions. The method accurately detects the impacts of the right and left legs, reducing the number of sensors traditionally needed for running assessment, thus enhancing user comfort and convenience especially in competitive events such as the marathon. This method is more accurate in identifying the right and left side impacts, especially during real-world runs. In such scenarios, the acceleration waveform can significantly differ from those in controlled environments.

According to one embodiment of the invention, the method is implemented using a portable IMU 116 shown in FIG. 1B. The IMU includes an accelerometer 104, rate gyroscope 106, digital processor 108, memory 110, battery 112, and radio 114. The accelerometer 104 and rate gyroscope 106 measure movements and provide a data stream of measurements to digital processor 108 and memory 110. A battery 112 provides power to the components of the device and radio 114 is provided for communicating the data and/or results of processing to other devices, e.g., using a wireless Bluetooth connection.

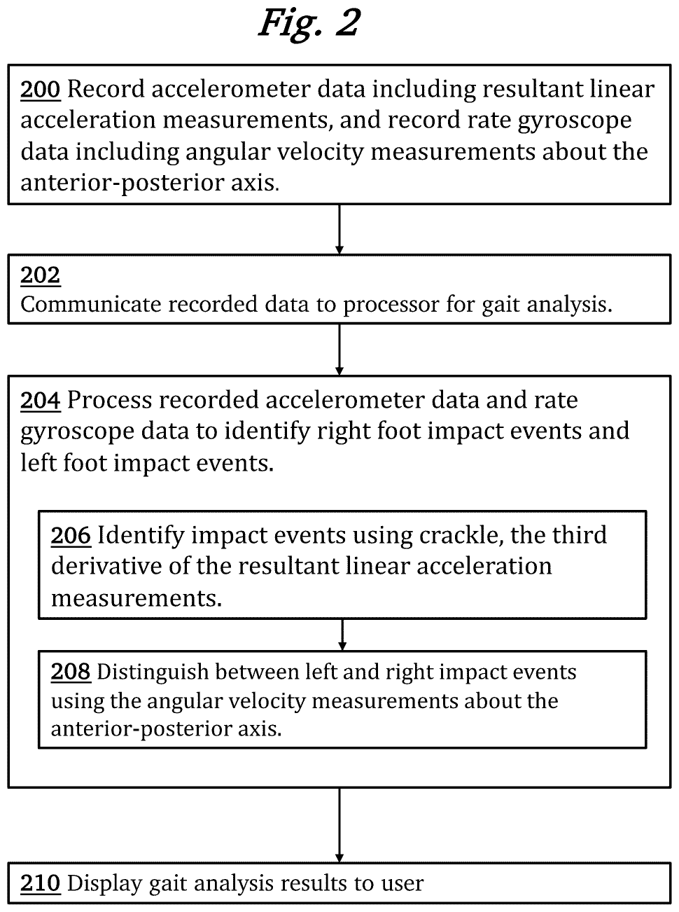

As illustrated in FIG. 2, the IMU device implements a method for biomechanical motion tracking. In step 200 accelerometer and rate gyroscope data are recorded from the IMU mounted on the sacrum of a runner during running. The accelerometer data includes a time data stream of resultant linear acceleration measurements. The rate gyroscope data includes a time data stream of angular velocity measurements about the anterior-posterior axis of the runner. In step 202 the recorded data is communicated to the processor for gait analysis. In embodiments where a single device both records the data and processes the data, the communication to the processor is an on-board communication within the same device. Alternatively, in embodiments where one device records the data and a separate device performs the processing, the communication can be a wired or wireless data communication from the recording device to the processing device. In step 204 recorded accelerometer data and rate gyroscope data are processed to identify right foot impact events and left foot impact events. It is important to note that the processing uses acceleration and angular velocity data from only a single IMU and does not rely on measurements from any other IMUs positioned at other locations on the runner. In addition, the single IMU is mounted on the sacrum of the runner. No IMU is required at other body locations such as the feet or legs. In step 210 the results of the processing, which may include other gait analysis results in addition to left and right impact events, are displayed either by the IMU itself or on another device that has received the results from the IMU device, e.g., over a wireless Bluetooth connection.

The processing step 204 includes a first sub-step 206 of identifying impact events using crackle, the third derivative of the resultant linear acceleration measurements, and a second sub-step 208 of distinguishing between left and right impact events using the angular velocity measurements about the anterior-posterior axis.

In sub-step 206 impact events are identified by computing the crackle, i.e., the third derivative with respect to time of the resultant linear acceleration time series measurement data, and identifying maxima of the computed crackle. The times of these maxima are then used to identify peaks in the resultant linear acceleration that are closest temporally to the locations of the crackle maxima. These peaks are the identified foot impact events.

In sub-step 208 left and right impact events are distinguished. To determine whether a particular impact event identified in step 206 is a right foot impact or a left foot impact, a search for extrema (i.e., peaks or troughs) in the angular velocity data is performed within a time window of 5 ms around the time of the identified impact event. The impact event is identified as a right foot impact or left foot impact depending on whether the angular velocity extremum in the window is positive or negative, respectively.

The method was tested with the participation of ten healthy recreational runners. Participants were equipped with a multi-axis IMU (Casio, Tokyo, JPN) positioned approximately on the sacrum, clipped to participant's waistband. The IMU sensors recorded three-dimensional linear accelerations and angular velocities at 200 Hz to produce time series data streams. The data were post-processed using a Kalman filter to align the vertical axis of the local IMU coordinate system with gravity. For testing purposes, each participant ran on level ground (LG), incline (IN), and decline (DE) at an angle of +7.5 degrees. The protocol included thirteen 30-second trials: five runs at LG; three paces slower than 5k race pace, one at 5k race pace, and one optional trial faster than 5k race pace. The same four initial runs at LG were then repeated at IN and DE. The total range of speeds was from 3.16 to 4.88 m/s.

The third derivative of the acceleration is referred to as crackle. For example, if the acceleration function is represented as an n-th degree polynomial A(t)=aₙtⁿ+aₙ₋₁tⁿ⁻¹+ . . . +a₁t+a₀, the crackle function will be represented as the polynomial A′″(t)=aₙn(n-1)(n-2)tⁿ⁻³+aₙ₋₁(n-1)(n-2)(n-3)tⁿ⁻⁴+ . . . +a₄t+a₃.

The crackle is calculated from the measured resultant acceleration (a_{mag}) data. The impact identification process involves detecting the critical points where the crackle becomes zero, indicating potential peaks or troughs. Within a 10 ms window around each a_{mag} peak, points where the crackle is zero are found. These critical points correspond to the moments of extremum in the crackle function.

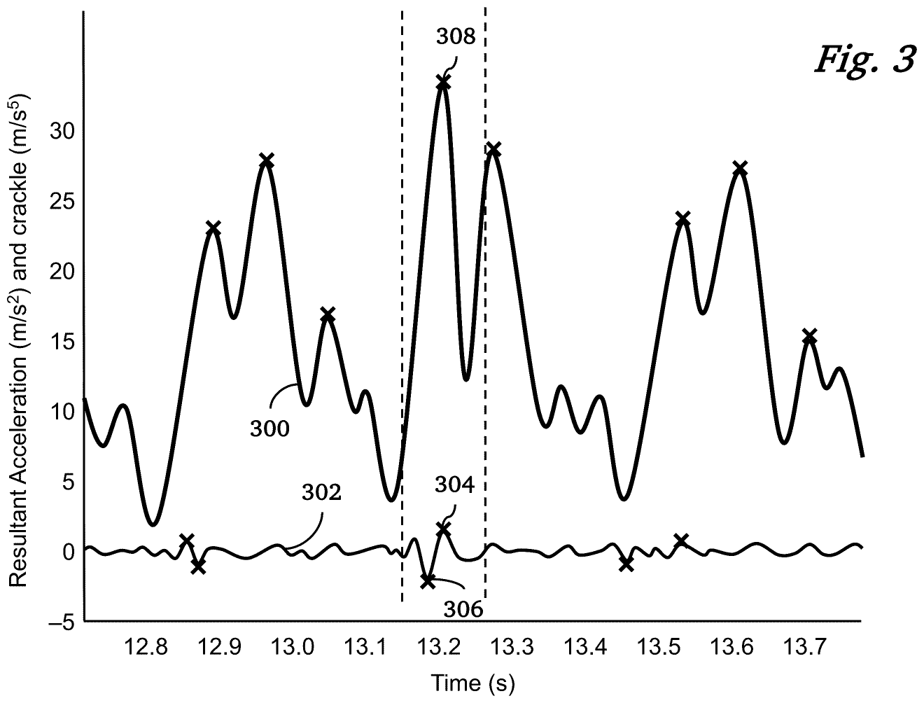

FIG. 3 is a graph of a resultant acceleration data 300 and corresponding crackle data 302 derived from experimental data. Vertical lines indicate the edges of time windows around the crackle extrema where a closest peak in the acceleration is found. As an example, extrema 304 and 306 are identified in the crackle data near 13.2 seconds. Within a 0.1 s window around this time, there is a peak 308 in the acceleration data, which is identified as a foot impact event.

To identify the extrema in the acceleration data, we use the built-in findpeaks function in MATLAB. This function detects local maxima based on a defined minimum peak height and minimum peak distance (i.e., the minimum time window between adjacent peaks). These parameters help reduce noise and ensure only meaningful peaks are captured.

The threshold for the minimum peak height is based on prior studies and previous data. Since we know the typical range of acceleration magnitudes during foot impacts, we use this knowledge to set a realistic threshold; effectively limiting the number of peaks detected and focusing the analysis on the most relevant ones. We also set the minimum peak distance based on the expected time between steps.

Based on our observations to capture peaks during foot impact, the minimum peak height is preferably selected to be 0.6×max (a_{mag}). Based on previous studies, the minimum peak distance has a temporal window of 0.2 second. Default ranges may include a minimum peak height of 1.5-5 m/s² and a time window between 0.12-0.2 seconds.

Once the acceleration peaks are identified, we compute the corresponding crackle signal by successively applying the MATLAB diff function three times to the acceleration data. Around the time of foot impact, the crackle signal consistently shows the highest-magnitude extrema (both positive and negative) due to the rapid change in acceleration. We then apply an additional threshold to the crackle signal, isolating only those peaks with the largest magnitudes. In one embodiment, the minimum peak height threshold is preferably 0.45 and the minimum peak distance threshold is 0.2 second.

Finally, we define a short window of ±0.05 seconds around each high-magnitude crackle peak to identify the closest impact peak in the acceleration signal to the peak in crackle. This ensures isolation of foot peak impacts even if they have lower amplitude than the mid-stance peak in the resultant acceleration.

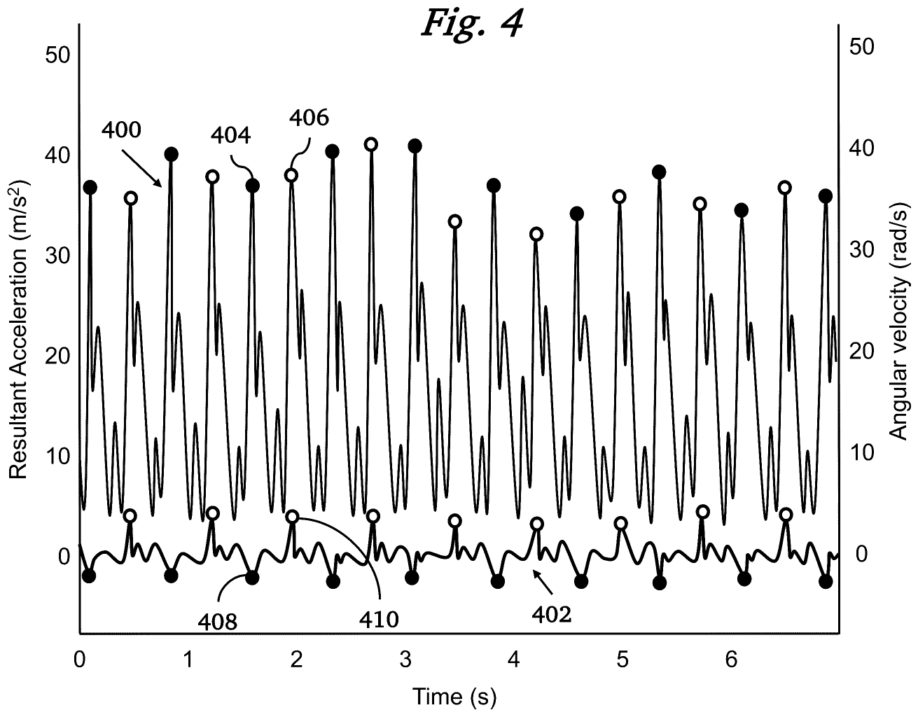

For each peak in a_{mag} that is found, the technique assesses the corresponding angular velocity (ωy) peak or trough. If the one found is a maximum, then the a_{mag} peak is happening during the right impact and vice versa. To illustrate this, FIG. 4 shows graphs of acceleration 400 and angular velocity 402. Acceleration peaks 404 and 406 correspond to angular velocity minimum 408 and angular velocity maximum 410, which allows identification of peak 404 as a left foot impact event and peak 406 as a left foot impact event. In this way, right and left peaks in resultant linear acceleration a_{mag} are distinguished using the maximum and minimum Sacral angular velocity ωy.

This approach to identifying right and left gait events is particularly effective in capturing the initial peak in acceleration data during foot impact with the ground, often overlooked by other methods. The tests demonstrate that the present method can accurately identify and distinguish right and left impacts during level ground, incline and decline runs across a range of speeds. The results are compared with other existing methods to highlight its effectiveness.

Compared with existing methods, the present technique is more robust and adaptable to changes in running patterns because it treats each peak in the acceleration data independently from all others. The algorithm identifies each peak in the a_{mag} data and then uses the ‘crackle’ function to refine the peak detection. The advantage of using ‘crackle’ over jerk, or snap lies in its sensitivity to rapid changes in acceleration, which are characteristic of the initial impact in a running cycle. While jerk, and snap can provide valuable information about the overall pattern of movement, ‘crackle’ offers a more nuanced view of the intricate changes occurring at each moment of impact. This makes ‘crackle’ particularly effective for capturing the initial peak in the acceleration data, providing a more accurate representation of the running cycle.

By treating each peak independently, the algorithm can adapt to changes in the running pattern, making it particularly effective for analyzing outdoor running where various events can interrupt the running cycle. This peak-based approach provides a more dynamic classification of right and left impacts, improving the accuracy of the follow-on analyses required for gait analysis.

The combination of crackle-based peak detection with angular velocity-based right-left discrimination enhances the robustness of the analysis, ensuring the accurate identification of right and left impacts, particularly crucial in complex running scenarios.

In comparison to other methods, this technique demonstrated better accuracy in identifying the side of ground contact during running on level ground (LG). The mean accuracy was 99.2%. This is significantly higher than other methods, which have an accuracy of less than 82% and are limited to specific running conditions.

Table 1. Accuracy in finding right and left impact in level ground, decline, and incline, comparing the current method with existing methods reported by Lee, Benson, and Auvinet.

Accuracy (%)

| Method | Level Ground | Decline | Incline |
|---|---|---|---|
| Current Method | 99.2 | 99.8 | 90.8 |
| Lee | 81.9 | — | — |
| Benson | 54.6 | — | — |
| Auvinet | 75 | — | — |

The present method also performed well in more challenging conditions. In decline conditions, the mean accuracy was 99.8%. In incline conditions, the mean accuracy was 90.8%. Despite the decrease in accuracy in incline conditions, the method still outperformed the other methods.

This technique allows the use of a single inertial sensor, strategically placed on the sacrum, as an effective tool for accurately and reliably detecting right and left impact during running in diverse conditions. In addition to identifying right and left impact events, the sensor can also process the acceleration and angular velocity data to determine other gait events, including initial contact and toe-off events. The left and right foot strike events can then be used to compute other gait features such as cadence and stride length. With the present method of detecting foot impact events using a sacrum-mounted IMU, we can now compute and gather all the information that was traditionally obtained from foot-mounted IMUs. This includes stride length (the distance covered in one full step cycle, from one foot strike to the next same foot strike), step length (the distance between the heel strike of one foot and the heel strike of the other foot, cadence (the number of steps taken per minute), stance time (the time each foot spends in contact with the ground during walking or running), estimate of ground reaction forces (the force exerted by the ground on the foot during walking or running), pelvic tilt (the angle of the pelvis relative to the horizontal plane), pelvic rotation (the twisting movement of the pelvis around a vertical axis), and running symmetry (the balance between the movements of the left and right sides of the body during running),

Placing the IMU near the sacrum, which is near the body's center of mass, provides accurate data on the body's orientation and movement, which is crucial for maintaining balance and stability in robotic systems. This placement allows for more natural and precise tracking of human movements, which can be beneficial for applications in exoskeletons and assistive robots. Clipping the IMU to a waistband minimizes interference from other body parts, ensuring cleaner and more accurate data.

This method will enable a deeper exploration of running biomechanics across various terrains like level ground (LG), inclines (IN), and declines (DE), ultimately aiding in the prevention and management of running-related injuries. By providing a more accurate understanding of the forces involved in running, this method can contribute to better injury prevention strategies. The detailed analysis of foot impacts can also be used to improve running techniques, potentially enhancing athletic performance. The IMU could be incorporated into wearable technology for runners, providing real-time feedback on running technique through integration with fitness tracking devices or apps. For fitness applications, an IMU in this position can accurately track exercises and provide feedback on form and technique. Using an IMU near the sacrum can provide detailed insights into gait patterns, which is useful for medical diagnostics and treatment planning. This setup can help in monitoring and correcting posture, which is beneficial for both health and ergonomic applications. By analyzing movement and posture, an IMU can help assess ergonomic risks in the workplace. This data can be used to design better workstations and reduce the risk of musculoskeletal disorders. Continuous monitoring of posture and movement can help identify patterns that may lead to injury or strain. This information is valuable for creating interventions to improve worker health and productivity.

The device could also be integrated and applied in various other contexts including motion capture and biomechanically accurate animation, real-time adaptive movement for gaming, VR player movement mapping, and data-driven character development. A single IMU clipped to the waistband can help in monitoring and assisting with human movements, providing support for rehabilitation or enhancing physical capabilities. For sports and physical therapy, an IMU in this position can capture detailed motion data, helping in performance analysis and injury prevention. In collaborative robots, an IMU near the sacrum can help the robot understand and predict human movements, improving safety and efficiency in shared workspaces. In VR/AR applications, this IMU placement can enhance the realism of avatars by accurately mapping the user's movements to the virtual environment.

1. A method for biomechanical motion tracking, the method comprising:
a) recording accelerometer and rate gyroscope data from an inertial measurement unit mounted on the sacrum of a runner, wherein the accelerometer data comprises resultant linear acceleration measurements and the rate gyroscope data comprises angular velocity measurements about the anterior-posterior axis;
b) processing the recorded accelerometer data to identify right foot impact events and left foot impact events, wherein the processing comprises identifying impact events using the third derivative of the resultant linear acceleration measurements, and distinguishing between left and right impact events using the angular velocity measurements about the anterior-posterior axis, wherein the processing uses acceleration and angular velocity data from only the inertial measurement unit mounted on the sacrum of the runner.

2. The method of claim 1 wherein identifying impact events using the third derivative of the resultant linear acceleration measurements comprises using maxima of the third derivative of the resultant linear acceleration measurements to determine locations of impact peaks in the resultant linear acceleration that are closest temporally to the locations of the crackle maxima.

3. The method of claim 1 wherein distinguishing between left and right impact events comprises searching for extrema in the frontal plane angular velocity within a window of 5 ms around an identified impact event, and identifying the impact event as a right foot impact or left foot impact depending on whether the angular velocity peak is positive or negative.

4. The method of claim 1 wherein the recording and processing steps are performed by a single portable electronic device.

5. The method of claim 1 wherein the recording and processing steps are performed by two distinct portable electronic devices, and wherein the method further comprises communicating the recorded accelerometer and rate gyroscope data between the two distinct portable electronic devices.

6. The method of claim 1 further comprising processing the recorded accelerometer and rate gyroscope data, right foot impact events, and left foot impact events to provide analysis of gait patterns.

7. The method of claim 1 further comprising processing the recorded accelerometer and rate gyroscope data, right foot impact events, and left foot impact events to provide analysis of athletic technique.
